(19) United States
(12) Patent Application Publication
(10) Pub. No.: US 2017/0197047 A1
(43) Pub. Date: Jul. 13, 2017

(54) PORTABLE ELECTROMECHANICAL RESUSCITATOR BAG COMPRESSION DEVICE

(71) Applicant: Inertia Engineering + Design Inc., Toronto (CA)

(72) Inventors: Raymond John MINATO, Toronto (CA); Olakulehin Oladayo EMMANUEL, Brampton (CA); Manjunath ANAND, Bangalore (IN); Mollie Beauvais JAMESON, St. John's (CA); Daniel Nathan WOODSIDE, Thorndale (CA); Eleu Thereus Tai-Sung UM, Toronto (CA); Randy YANG, Oakville (CA); Ethan David MAIOLO, Niagara Falls (CA)

(21) Appl. No.: 15/401,696

(22) Filed: Jan. 9, 2017

Related U.S. Application Data
(60) Provisional application No. 62/276,551, filed on Jan. 8, 2016.

(51) Int. Cl. A61M 16/00; A61M 16/08; A61M 16/06

(52) U.S. Cl. CPC A61M 16/0078; A61M 16/0084; A61M 16/06; A61M 16/0875; A61M 16/0051; A61M 2205/33; A61M 2205/18; A61M 2205/8206; A61M 2205/502; A61M 2205/3606; A61M 2205/362; A61M 2209/08

(57) ABSTRACT

A portable device including a housing comprising a first opening and a second opening and a resuscitator bag. The resuscitator bag is disposed at least partially within the housing and includes an air inlet supported at the first opening of the housing, an air outlet supported at the second opening of the housing, and a self-inflating bag. The portable device also includes a double-sided compression mechanism disposed within the housing. The double sided-compression mechanism includes a pair of arms at least partially surrounding the self-inflating bag. The pair or arms are configured to move towards each other to compress the self-inflating bag to provide positive pressure ventilation via the air outlet and to move away from each other to enable re-inflation of the self-inflating bag via the air inlet; and, a motor coupled to the pair of arms for moving the pair of arms towards and away from each other.

### CROSS-REFERENCE TO RELATED APPLICATIONS

This application claims priority to U.S. provisional application 62/276,551, filed Jan. 8, 2016, which is incorporated herein by reference.

### FIELD

The present disclosure relates to a portable electromechanical resuscitator bag compression device for providing positive pressure ventilation to patients.

### BACKGROUND

Ventilators and self-inflating resuscitator bags are utilized to provide positive pressure ventilation to patients that are unable to breath on their own. Ventillators are generally used in hospitals while self-inflating resuscitator bags are generally used when a patient is being treated in the field or in transport to a hospital. Most ventilators are not generally suitable for field as these devices are not portable. In resource limited countries, ventilators are not widely available, due to their high cost, and so medical personnel and sometimes family members have no alternative but to continuously manually compress a resuscitator bag to help patients to breath for long periods of time—from days to weeks at a time. Self-inflating resuscitator bags are manually compressed by one or more hands of medical personnel to provide positive pressure ventilation to a patient. One limitation of self-inflating resuscitator bags is that the manually compression of these bags renders it difficult for the medical personnel operating the bag to perform additional life-saving tasks, such as, for example, cardiopulmonary resuscitation (CPR). Another limitation of self-inflating resuscitator bags is that the manual compression of such bags by medical personnel is both fatiguing and renders it challenging for the medical personnel to maintain a consistent rhythm when compressing the bag. It is important for medical personnel, when operating self-inflating resuscitator bag, to maintain a consistent rhythm of compressions to mimic the normal rhythm of a person's breathing.

### SUMMARY

According to one aspect, there is provided a portable electromechanical resuscitator bag compression device including a housing comprising a first opening and a second opening and a resuscitator bag. The resuscitator bag is disposed at least partially within the housing and includes an air inlet supported at the first opening of the housing, an air outlet supported at the second opening of the housing, and a self-inflating bag. The portable electromechanical resuscitator bag compression device also includes a double-sided compression mechanism disposed within the housing. The double sided-compression mechanism includes a pair of arms at least partially surrounding the self-inflating bag. The pair or arms are configured to move towards each other to compress the self-inflating bag to provide positive pressure ventilation via the air outlet and to move away from each other to enable re-inflation of the self-inflating bag via the air inlet; and, a motor coupled to the pair of arms for moving the pair of arms towards and away from each other.

The self-inflating bag can float in between the pair of arms of the double-sided compression mechanism.

A first arm of pair of arms may face a first side of the self-inflating bag and a second arm of the pair or arms may face a second side of the self-inflating bag.

The first arm comprises a first layer of frictionless material for reducing wear on the first side of the self-inflating bag and the second arm comprises a second layer of frictionless material.

The first arm may have a cam shape to reduce wear on the first side of the self-inflating bag and the second arm may have a cam shape to reduce wear on the second side of the self-inflating bag.

The first arm may include a first hook coupled to a first hoop on the first side of the self-inflating bag and the second arm may include a second hook coupled to a second hoop on the second side of the self-inflating bag for controlling re-inflation of the self-inflating bag when the first arm and the second arm move away from each other.

The portable electromechanical resuscitator bag compression device may further include an input device for inputting an inhale and exhale rate for the positive pressure ventilation.

The portable electromechanical resuscitator bag compression device may further include a processor in communication with the input device and may be configured to: receive the inhale and exhale rate from the input device; determine a rate of compression for the double-sided compression mechanism corresponding to the inhale and exhale rate; control the motor for controlling movement of the pair of arms towards and away from each other at a rate of compression corresponding to the inhale and exhale rate to provide positive pressure ventilation via the air outlet at a number of breaths per minute corresponding to the inhale and exhale rate.

The first arm may include a first pressure sensor for measuring a force applied to the first side of the self-inflating bag and the second arm may include a second pressure sensor for measuring a force applied to the second side of the self-inflating bag as the pair of arms move towards and away from each other.

The portable electromechanical resuscitator bag compression device may further include a power supply disposed in the housing for supplying power to the processor and the motor.

The portable electromechanical resuscitator bag compression device may further include a power switch coupled to the power supply for connecting and disconnecting the power supplied by the power supply to the processor and the motor.

The housing may include a front cover comprising a handle for lifting the portable electromechanical resuscitator bag compression device; and a back cover comprising a recess configured for grasping by a hand of a user to facilitate lifting of the portable electromechanical resuscitator bag compression device.

The first side cover of the pair of side covers may include vents for circulating air into the housing and for dissipating heat from within the housing.

The portable electromechanical resuscitator bag compression device may further include a strap coupled to each side cover and extending over the top cover to facilitate carrying the portable electromechanical resuscitator bag compression device.

The portable electromechanical resuscitator bag compression device may further include an output device coupled to the processor for providing an audible output when operation of the portable electromechanical resuscitator bag compression device fails.

The portable electromechanical resuscitator bag compression device may further include a removable battery disposed in the housing for supplying power to the processor and the motor.

The portable electromechanical resuscitator bag compression device may further include: a power switch for activating and deactivating the power supply to discontinue supplying power to the processor and the motor.

### BRIEF DESCRIPTION OF THE DRAWINGS

Embodiments of the present invention will be described, by way of example, with reference to the drawings and to the following description, in which:

FIG. 1 is a front perspective view of a portable electromechanical resuscitator bag compression device in accordance with an implementation;

FIG. 2 is a rear perspective view of the portable electromechanical resuscitator bag compression device of FIG. 1;

FIG. 3 is a front perspective view of a portable electromechanical resuscitator bag compression device of FIG. 1 with a top cover of the portable electromechanical resuscitator bag compression device in an open position;

FIG. 4 is a partially cut away front perspective view of the portable electromechanical resuscitator bag compression device of FIG. 1;

FIG. 5 is a partially cut away side perspective view of the portable electromechanical resuscitator bag compression device of FIG. 1;

FIG. 6 is a perspective view of a double-sided compression mechanism of the portable electromechanical resuscitator bag compression device of FIG. 1;

FIG. 7 is a perspective view of a camshaft of the double-sided compression mechanism of FIG. 6;

FIG. 8 is a block diagram of the portable electromechanical resuscitator bag compression device of FIG. 1;

FIG. 9 is a perspective view of a portable electromechanical resuscitator bag compression device in accordance with another implementation;

FIG. 10 is a partial front view of a double-compression mechanism and a resuscitator bag of a portable electromechanical resuscitator bag compression device in accordance with another implementation.

### DETAILED DESCRIPTION

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

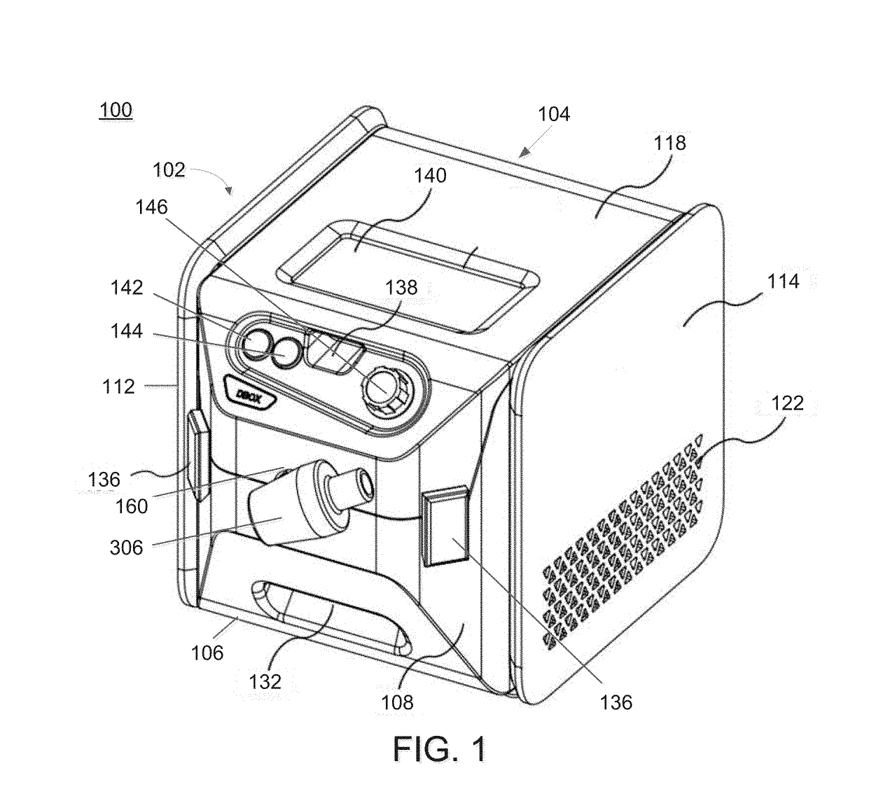

For the purposes of the present disclosure, the terms top, bottom, front, back, horizontal, and vertical are utilized herein to provide reference to the orientation of the device 100 when in use, as shown in FIG. 1 to FIG. 3.

The disclosure generally relates to a portable electromechanical resuscitator bag compression device for providing positive pressure ventilation to patients.

FIG. 1 to FIG. 3 show an example implementation of a portable electromechanical resuscitator bag compression device (referred to hereinafter as device 100) for providing positive pressure ventilation to patients. The device 100 includes a housing 102 with a generally rectangular cross-section. The housing 102 includes a top cover 104, a bottom plate 106, a front cover 108, back cover 110, and a first side cover 112 and a second side cover 114 opposing the first side cover 112. The top cover 104 of the housing 102 includes a front portion 116, a middle portion 118, and a back portion 120. The first side cover 112 and the second side cover 114 each extend from the middle portion 118 of the top cover 104 to the bottom plate 106. The first side cover 112 and second side cover 114 each include vents 122 for circulating outside air into the housing 102 and for dissipating heat from inside the housing 102.

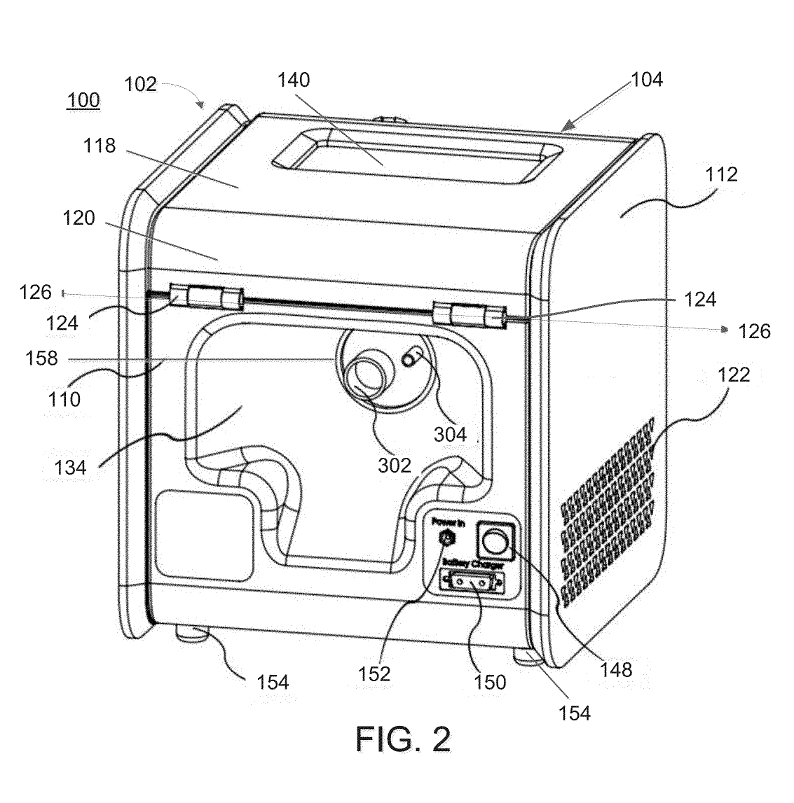

Although the first side cover 112 and second side cover 114 of the housing 102 of the device 100 of FIG. 1 and FIG. 2 include vents 122, in some alternative implementations only one of the first side cover 112 and second side cover 114 includes vents 122. In other alternative implementations, the vents 122 may be located at other suitable locations on the housing 102 of the device 100 for circulating outside air into the housing 102 and for dissipating heat from inside the housing 102. In still other alternative implementations, the device 100 may not include any vents 122.

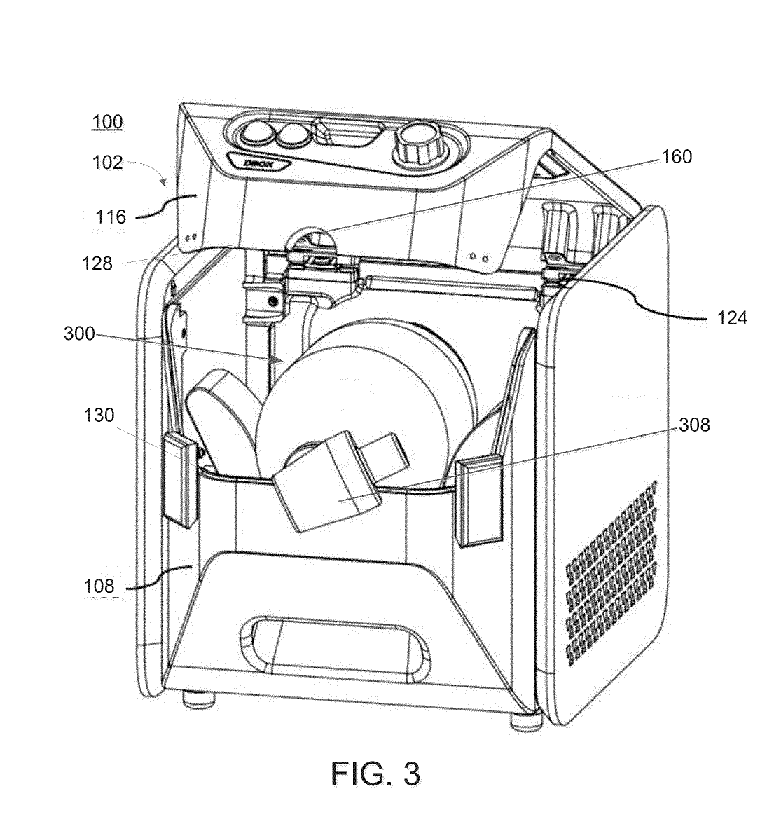

The front cover 108 of the housing 102 extends from the bottom plate 106 to the front portion 116 of the top cover 104. The back cover 110 of the housing 102 extends from the bottom plate 106 to the back portion 120 of the top cover 104. The back cover 110 and the back portion 120 of the top cover 104 are attached to each other by a pair of hinges 124 (referred to hereinafter collectively as hinges 124 and individually as hinge 124). The hinges 124 enable the top cover 104 to rotate about a horizontal axis 126 (FIG. 2) that extends between hinges 124 in order to open and close the device 100. The top cover 104 is rotatable about the horizontal axis 126 from a closed position (see FIG. 1) in which an edge 128 of the front portion 116 of the top cover 104 abuts an edge 130 of the front cover 108 to an open position (FIG. 3) in which the edge 128 of the front cover 108 is spaced from the edge 130 (FIG. 3) of the front portion 116 of the top cover 104. As shown in FIG. 3, when the top cover 104 is in the open position, the resuscitator bag 300 may be removed from the housing 102 and replaced with a new resuscitator bag.

Although the back cover 110 and the back portion 120 of the top cover 104 of the housing 102 of the device 100 shown in FIG. 1 to FIG. 3 are attached to each other by a pair of hinges 124, in alternative implementations, other suitable number of hinges 124 may be utilized to attach the back cover 110 to the back portion 120 of the top cover 104. In still other alternative implementations, the back cover 110 and the back portion 120 of the top cover 104 may be attached to each other using any suitable joint device that enables the top cover 104 to rotate between the open and closed positions.

Referring again to FIG. 1 and FIG. 2, the front cover 108 of the housing 102 includes a handle 132 for grasping by one hand of a user and the back cover 110 includes a recess 134 that is shaped and dimensioned to be grasped by the other hand of the user. The handle 132 is integrally formed with the front cover 108 and extends from the bottom plate 106 at angle away from the front cover 108 such that the handle 132 is spaced from the front cover 108 to facilitate grasping of the handle 132 by a hand of a user. The recess 134 and the handle 132 facilitate lifting of the device 100 by a user.

Although the handle 132 of the device 100 is integrally formed with the front cover 108 in FIG. 1 and FIG. 2, in alternative implementations the handle 132 may be a separate piece attached to the front cover 108 at the bottom plate 106 of the housing 102 by fasteners, such as for example, nuts and bolts.

The front cover 108 includes a pair of locks 136 (referred to hereinafter collectively as locks 136 and individually as lock 136) for securely locking the front cover 108 to the the front portion 116 of the top cover 104 when the top cover 104 is in the closed position to inhibit the top cover 104 from opening. The locks 136 may be any suitable type of mechanical lock, such as, for example, draw latches, cam latches, and the like. It will be appreciated that in the example implementation shown in FIG. 1 to FIG. 3, the locks 136 must be unlocked to enable the top cover 104 to be opened.

Referring to FIG. 1, the front portion 116 of the top cover 104 includes a groove 138 shaped and dimensioned to receive a finger of a user to facilitate opening of the top cover 104. The front portion 116 of the top cover 104 also includes indicator lights 142, 144 and an input device 146 that are described in further detail below. The middle portion 118 of the top cover 104 includes a window 140 that enables viewing of the interior of the housing 102 of the device 100. As shown in FIG. 2, the back cover 110 of the housing 102 includes an power switch 148 for turning on and off the device 100, a battery charging port 150, and a power port 152 that are described in further detail below. The housing 102 also includes four legs 154 (referred to hereinafter individually leg 154 and collectively as legs 154) located at the four corners of the bottom plate 106 of housing 102. Each leg 154 extends substantially vertically away from an outer surface of the bottom plate 106 for resting the device 100 on a surface, such as, for example, a floor, a table, or the ground. It will be appreciated that although four legs 154 are shown in the implementation of FIG. 1 and FIG. 2, the device 100 may have any suitable number of legs 154 for resting the device 100 on a surface.

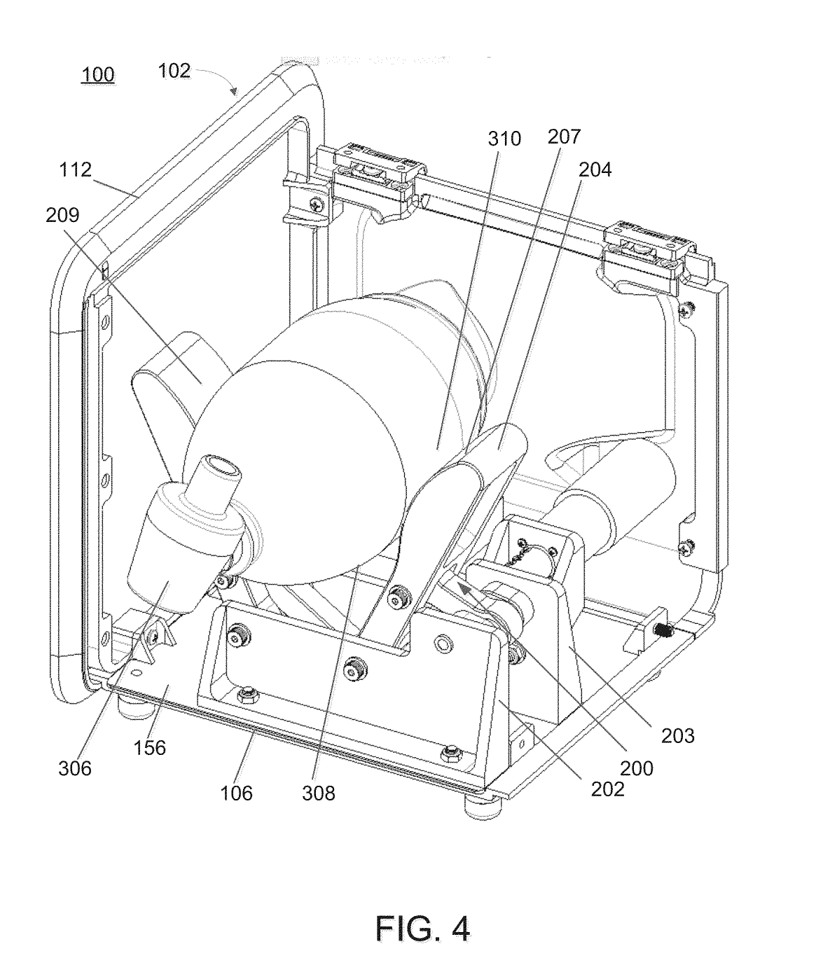

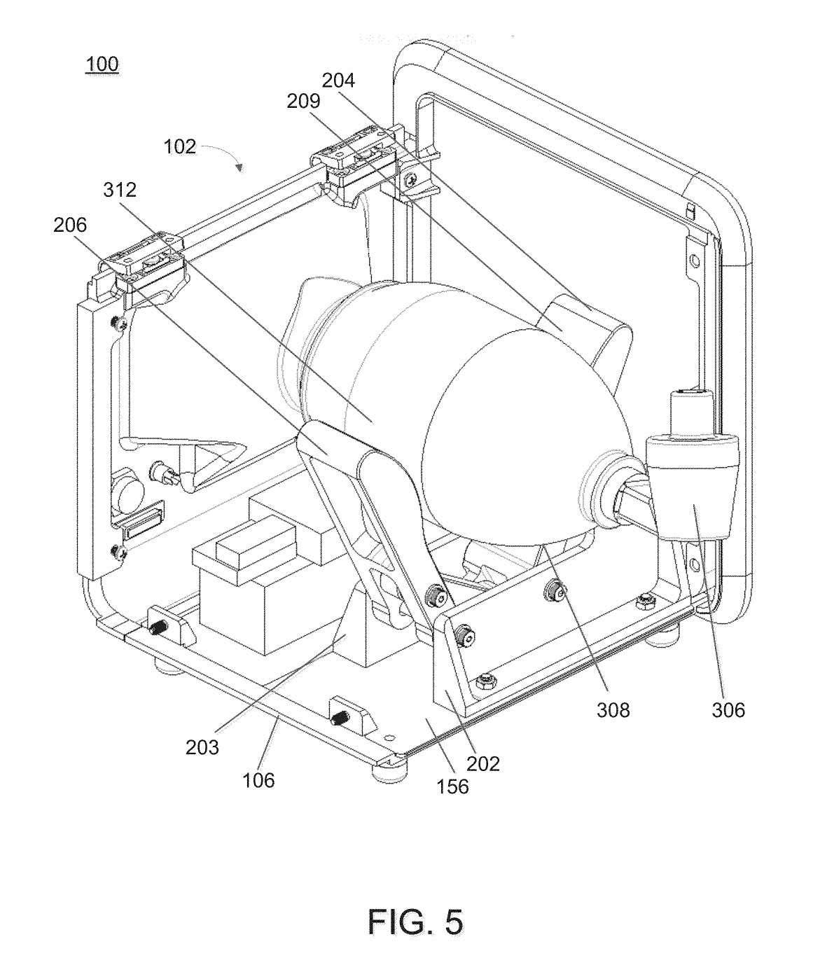

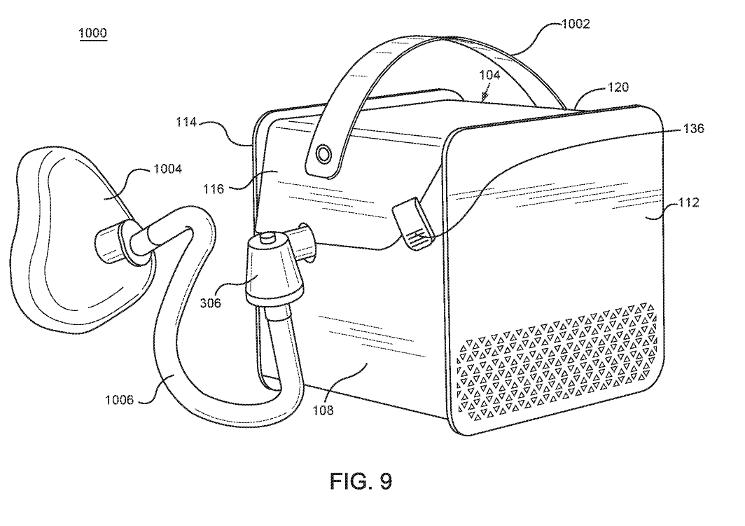

Referring now to FIG. 4 and FIG. 5, partially cutaway perspective views of the device 100 are shown. In FIG. 4 and FIG. 5, the top cover 104, the front cover 108, and the second side cover 114 are removed to depict internal components of the device 100. As shown in FIG. 4 and FIG. 5, the device 100 includes a double-sided compression mechanism 200 and a resuscitator bag 300. The double-sided compression mechanism 200 is disposed within the housing 102 of the device 100 and affixed to an inner surface 156 of the bottom plate 106 by chassis 202, 203. The resuscitator bag 300 is disposed at least partially within the housing 102. The resuscitator bag 300 includes an air inlet 302, an oxygen inlet 304 (FIG. 2) and air outlet 306 (FIG. 2), and a self-inflating bag 308. The air inlet 302 and the oxygen inlet 304 of the resuscitator bag 300 extends through a first opening 158 (FIG. 2) of the housing 102 and are supported at the first opening 158 (FIG. 2) of the housing 102. The first opening 158 (FIG. 2) is shaped and dimensioned to surround the air inlet 302 and the oxygen inlet 304. The air outlet 306 extends through a second opening 160 (FIG. 1) of the housing 102 and is supported at the second opening 160 (FIG. 1) of the housing 102. The oxygen inlet 304 is configured for connection to a hose (not shown) that attaches to an oxygen tank to provide oxygen to a patient. The air outlet 306 is configured to connect to a flexible hose (see FIG. 9) as described in further detail below.

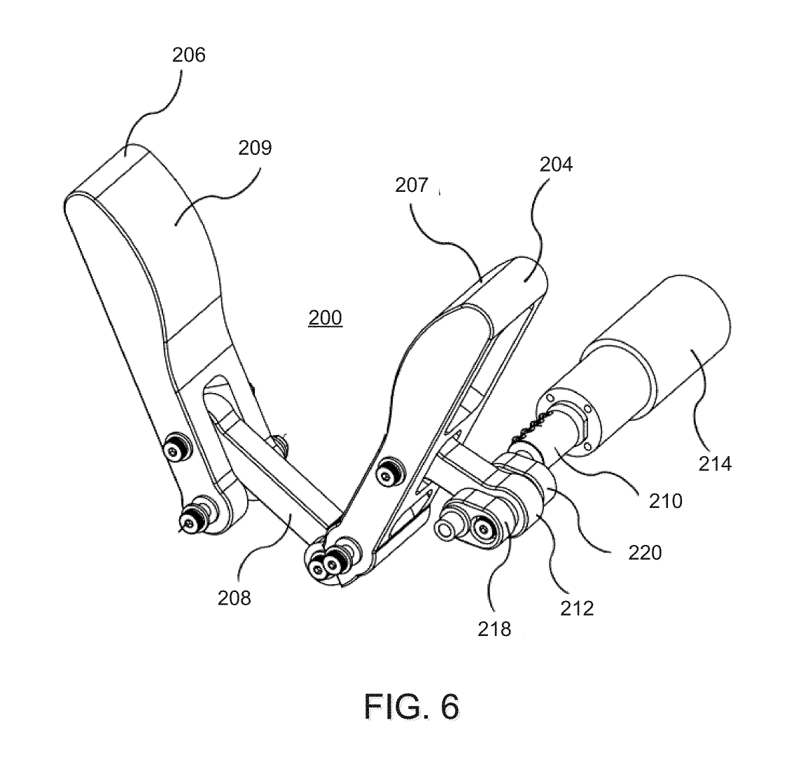

Referring now to FIG. 6, an example implication of the double-sided compression mechanism 200 is shown in isolation. The double-sided compression mechanism 200 include a pair of arms, including a driving arm 204 and a driven arm 206, that are coupled together by a connecting link 208. The driving arm 204 has a driving arm surface 207 that faces and is in contact with a first side 310 of the of the self-inflating bag 308 (see FIG. 4). Similarly, the 204 has a driven arm surface 209 that faces and is in contact with a second side 312 of the self-inflating bag 308 (see FIG. 5).

The driving arm 204 is coupled to a crankshaft 210 through piston link 212. The piston link 212 is coupled to a motor 214 via a coupler 216. The coupler 216 is configured to allow variations in a position and an orientation between the motor 214 and crankshaft 210. As the motor 214 rotates the crankshaft 210 displaces the piston link 212, which in turn displaces the driving arm 204. The connecting link 208 drives the driven arm 206 in the opposite direction to driving arm 204. The opposite movement of the driving arm 204 and the driven arm 206 causes the driving arm 204 and the driven arm 206 to move towards from each other. In other words, as the motor 214 rotates the crankshaft 210 through 180 degrees of rotation and the crankshaft 210 pushes the piston link 212 to move the driving arm 204 and driven arm 204 towards each other (e.g close the driving arm 204 and the driven arm 206), which causes the driving arm 204 and the driven arm 206 to concurrently compress the first and second sides 310, 312 of self-inflating bag 308. As the motor 214 rotates the crankshaft 210 through the remaining 180 degrees of rotation, the crankshaft 210 pulls the piston link 212 which causes the driven arm 204 and the driving arm 206 to move away from each other (e.g. the driving arm 204 and the driving arm 206 to open) to allow the self-inflating bag 308 to reinflate.

In the example implementation shown in FIG. 6, one end of the connecting link 208 is coupled to the driving arm 204 by fasteners, such as, for example, shafts and bolts. Similarly, the other end of the the connecting link 208 is connected to the driving arm 204 is coupled to the driven arm 206 by fasteners, such as such as, for example, shafts and bolts.

Referring again to FIG. 4 and FIG. 5, the double-sided compression mechanism 200 is affixed to the chassis 202, 203 by fasteners, such as nuts and bots. The driving arm 204 and the driven arm 206 extend away from the chassis 202, 203 along the first side 310 and the second side 312 of the self-inflating bag 308 and at least partially surround the self-inflating bag 308. The self-inflating bag 308, which is supported at the first opening 158 and second opening 160 floats in between the driving arm 204 and the driven arm 206 of the double-sided compression mechanism 200.

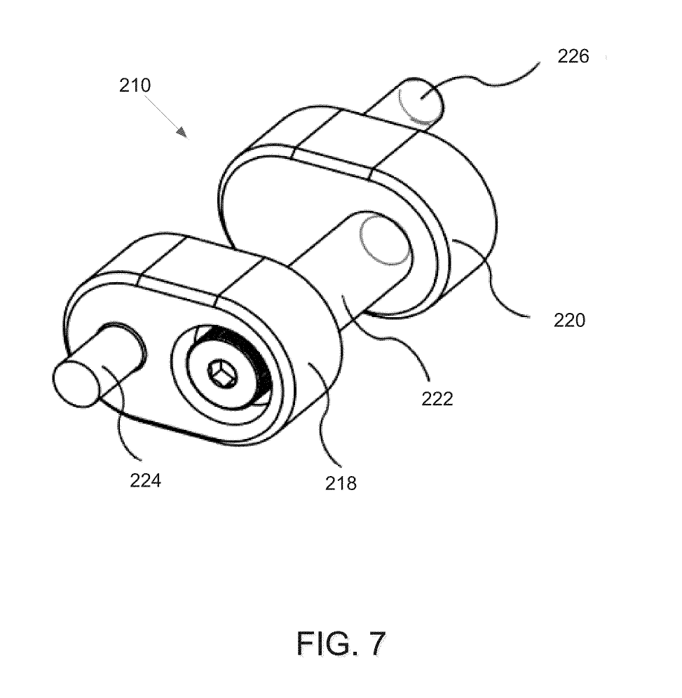

Referring now to FIG. 7, the crankshaft 210 of the double-sided compression mechanism 200 is shown in insulation. The crankshaft 210 converts rotational movement of the motor 214 into linear displacement of the driving arm 204 and the driven arm 206. The crankshaft 210 includes a first arm 218, a second arm 220, and a central shaft 222 that connects the first arm 218 and the second arm 220 together. The central shaft 222 also connects with piston link 212. The crankshaft 210 further includes a first shaft 224 that connects on one side to the first arm 218 to the chassis 202 and a second shaft 226 that connects the second arm 220 to the motor 214. The second shaft 226 has a rotational joint interface with the chassis 203 as depicted in FIG. 4.

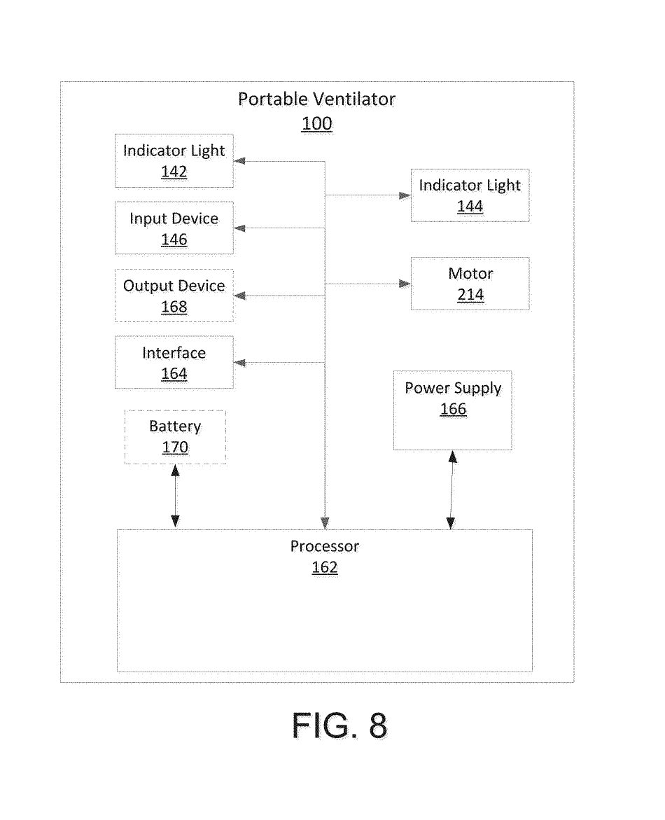

Referring to FIG. 8, a block diagram of the device 100 is shown. The device 100 includes multiple components, including a processor 162 that controls the overall operation of the device 100. The processor 162 is coupled to and interacts with other components, including the indicator lights 142, 144, the input device 146, a communication interface 164, a power supply 166, an output device 168 and the motor 214.

The input device 146 is configured to received input data. In the example implementation shown in FIGS. 1 to 3, the input device 146 is a rotary dial that is used to input an inhale and exhale rate for the positive pressure ventilation to be provided to a patient. The inhale and exhale rate corresponds to a number of breaths per minute to be provided to a patient by the device 100. The processor 162 receives the input data indicative of the inhale and exhale rate input using the rotary dial, determines a rate of compression for the double-sided compression mechanism 200 that corresponds to the inhale and exhale rate, and controls the motor 214 to provide positive pressure ventilation to a patient via the air outlet 306 at the inhale and exhale rate that corresponds to the number of breaths per minute to be provided to the patient. For the purposes of the present disclosure, a rate of compression for the double-sided compression mechanism 200 is defined as a speed at which the pair of arms of double-sided compression mechanism 200 move towards and away from each other, in cycles per minute, to maintain a consistent rhythm of compressions of the self-inflating 308 to mimic the normal rhythm of a person's breathing.

Although the input device 146 in the illustrated implementation is a rotary dial, in other implementations, the input device 146 may be a mechanical button, a touchscreen display comprising a graphical user interface with one or more selectable buttons or options. Each selectable button or option is associated with a inhale and exhale rate that corresponds to a number of breaths per minute to be provided to the patient to enable a user to input a desired number of breaths per minute to be provided to the patient.

The processor 162 is configured to interact with the indicator lights 142, 144 during operation of the device 100. The processor 162 activates indicator light 142, which is preferably red in color, when operation of the device 100 fails. The processor 162 activates indicator light 144, which is preferably green/blue in color, when the device 100 is operating normally.

The processor 162 is further configured to interact with communication interface 164 (interchangeably referred to interchangeably as interface 164), which may be implemented as one or more radios and/or connectors and/or network adaptors, configured to wirelessly communicate with one or more communication networks (not depicted). It will be appreciated that interface 164 is configured to correspond with network architecture that is used to implement one or more communication links to the one or more communication networks, including but not limited to any suitable combination of USB (universal serial bus) cables, serial cables, wireless links, cell-phone links, cellular network links (including but not limited to 2G, 2.5G, 3G, 4G+ such as UMTS (Universal Mobile Telecommunications System), GSM (Global System for Mobile Communications), CDMA (Code division multiple access), FDD (frequency division duplexing), LTE (Long Term Evolution), TDD (time division duplexing), TDD-LTE (TDD-Long Term Evolution), TD-SCDMA (Time Division Synchronous Code Division Multiple Access) and the like, wireless data, Bluetooth links, NFC (near field communication) links, WLAN (wireless local area network) links, WiFi links, WiMax links, packet based links, the Internet, analog networks, the PSTN (public switched telephone network), access points, and the like, and/or a combination. In some implementations, the processor 162 communicates, via the interface 164, with global positioning satellites (GPS) to obtain GPS coordinates of the device 100. In some other implementations, the processor 162 communicates, via the interface 164, with a network (not shown) to transmit the GPS coordinates of the device 100.

The power supply 166 powers components of device 100 including, but not limited to, indicator lights 142, 144, input device 146, processor 162, interface 164. Power supply 166 may include, a battery, a power pack and the like; however, in other implementations, power supply 166 connects to a mains power supply and/or a power adaptor (e.g. and AC-to-DC (alternating current to direct current) adaptor) via power port 152.

In some implementations, the device 100 also includes a battery 170 that supplies power to the motor 214 when an external power is not received or available from a mains power supply and/or power adaptor via the power port 152. In other implementations, the battery 170 is a rechargeable battery that is chargeable using the battery charging port 150. In still other implementations, the battery 170 is removable so that the battery 170 can be replaced when fully discharged.

In some implementations, the processor 162 is configured to interact with the output device 168 to provide an audible output when operation of the device 100 fails.

The operation of the device 100 will now be described. When the processor 162 receives input data from the input device 146 indicative of an inhale and exhale rate corresponding to a number of breaths per minute, the processor 162 determines a rate of compression that corresponds to the inhale and exhale rate. The processor 162 controls the motor 214 to rotate at the rate of compression that corresponds to the inhale and exhale rate, which causes the driving arm 204 and the driven arm 206 move towards and away from each other from a retracted position in which self-inflating bag 308 is fully inflated to a compressed position in which the self-inflating bag 308 is compressed and air is expelled through the air outlet 306. As the driving arm 204 and the driven arm 206 move towards each other, the driving arm surface 207 of the driving arm 204 applies a force to the first side 310 of the self-inflating bag 308 and the driven arm surface 209 of the driven arm 206 applies a force to the second side 312 of the self-inflating bag 308, which causes the self-inflating bag 308 to compresses and expel air through the air outlet 306. As the driving arm 204 and the driven arm 206 move away each other, the force applied to the first side 310 and the second side 312 of the self-inflating bag 308 is released and the self-inflating bag 308 reinflates as air is drawn into the self-inflating bag 308 through the air inlet 302. Optionally, oxygen may be provided to a patient by attaching a hose between the oxygen inlet 304 and an oxygen tank.

Referring now to FIG. 9, another example implementation of a portable electromechanical resuscitator bag compression device is shown. The portable electromechanical resuscitator bag compression device 1000 (referred to hereinafter as device 1000) is similar to the device 100, however, the housing 102 does not include the handle 132 and the back cover 110 does not include the recess 134. Rather, in this implementation, the device 1000 includes a strap 1002 attached to at first end to the first side cover 112 and at a second end to the second side cover 114 proximate the top cover 104. The strap 1002 extends over the top cover 104 to provide a handle for lifting the device 1000. The strap 1002 is configured to facilitate hand carrying of the device 1000. As depicted in FIG. 9, the air outlet 306 connects to the patient mask 1004 through a flexible hose 1006. The flexible hose 1006 is corrugated and allows the breath to be delivered to a mask that is applied over the patient's nose and mouth. The flexible hose 1006 also allows for the resuscitator bag 300, which is disposed within the device 1000, to be located remotely from the patient,. Moreover, the flexible hose 1006 also allows the resuscitator bag 300 to be separated from the patient to allow additional freedom of motion and comfort to the patient. The flexible hose 1006 allows the variation of distance between the device 1000 and a patient without affecting the air volume and pressure provided to the patient.

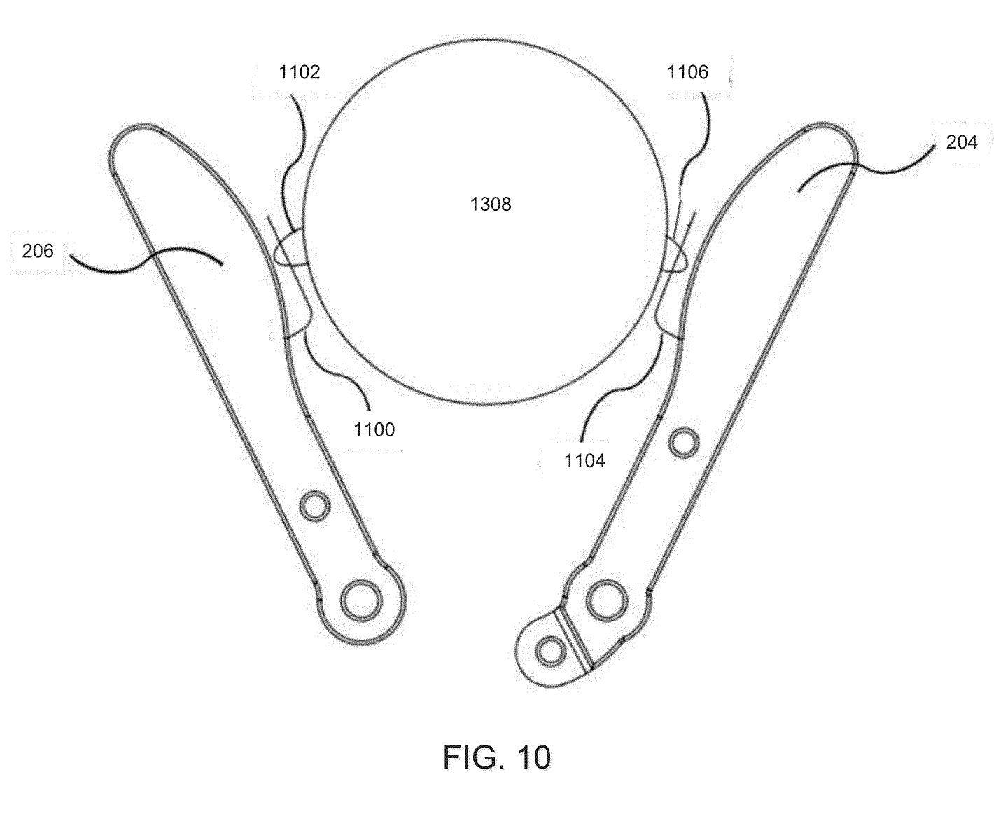

Referring now to FIG. 10, another implementation of the double-sided compression mechanism 200 and the resuscitator bag 300 are shown. In this implementation, only the driving arm 204 and the driven arm 206 of the double-sided compression mechanism 200 are shown. Similarly, only the self-inflating bag 308 of the resuscitator bag 300 is illustrated.

In the example implementation shown in FIG. 10, the driving arm 204 further includes a first open hook 1100 and the first side 310 of the self-inflating bag 308 includes a first hoop 1102. The first hoop 1102 is bonded to a first side 310 of self-inflating bag 308. The driven arm 206 includes a second open hook 1104 and the second side 312 of the self-inflating bag 308 includes a second hoop 1106. The second hoop 1106 is also bonded to a second side 312 of the self-inflating bag 308. The first open hook 1100 engages the first hoop 1102 and the second open hook 1104 engages the second hoop 1106 such that when the driving arm 204 and the driven arm 206 move away from each other, the first open hook 1100 pulls on the first hoop 1102 and the second open hook 1104 pulls on the second hoop 1106, to control a rate of reinflation of the self-inflating bag 308. Controlling the rate of reinflation of the self-inflating bag 308 ensures that the rate of compression corresponds to the breaths per minute correspond to the breaths per minute to be provided a patient which was inputted using the input device 146.

Although the device 100 in FIG. 1 to FIG. 3 includes a window 140 in the middle portion 118 of the top cover 104, in alternative implementations, the device 100 may be is made from a transparent material to enable viewing of the interior of the housing 102 of the device 100. In still other implementations, the top cover 104 of the device 100 may be is made from opaque material.

It will be appreciated that in the example implementation shown in FIG. 1 to 4, the bottom plate 106, the front cover 108, back cover 110, the first side cover 112 and the second side cover 114 are manufactured as separate pieces and are attached together using fasteners, such as, for example, nuts and bolts in order to form the housing 102. In other implementations, the front cover 108, the back cover 110, the first side cover 112 and the second side cover 114 are manufactured as a single piece.

In some implementations, the driving arm surface 207 has a cam shape to reduce wear on the first side 310 of the self-inflating bag 308 and the driven arm surface 209 each have a cam shape to reduce wear on the second side 312 of the self-inflating bag 308. In other implementations, the driving arm surface 207 and the driven arm surface 209 are each coated with a layer of frictionless material, such as for example, Telfon™. The layer of frictionless material on the driving arm surface 207 of the driving arm 204 reduces wear on the first side 310 of the self-inflating bag 308. Similarly, the layer of frictionless material on the driven arm surface 209 of the driven arm 206 reduces wear on the second side 312 of the self-inflating bag 308.

In some implementations, pressure sensors (not shown) are mounted on the driving arm surface 207 and/or the driven arm surface 209. The pressure sensor (not shown) are configured to measure a compression force applied to the first side 310 and the second side 312 of self-inflating bag 308 by the driving arm 204 and the driven arm 206. The pressure sensors (not shown) send the compression force measurements to the processor 162, which processes the compression force measurements to determine whether something is obstructing the normal function of the device 100 For example, the compression force measurements may indicate if the resuscitator bag 300 is unable to provide positive pressure ventilation (e.g deliver air) to a patient due to an obstruction in the patient's breathway or because the patient is choking, and the processor 162 may provide a visual output via indicator 142 and/or an audible output via the output device 168 to indicate that operation of device 100 has failed. Alternatively, the compression force measurements may indicate a mechanical malfunction in device 100, such as a leak in the self-inflating bag 308, and the processor may alert the medical staff of the failure via the indicator light 142 and/or the output device 168.

In some implementations, the device 100 includes a safety switch (not shown) coupled to the processor 162. The safety switch (not shown) is disposed on the front cover 108 such that when the top cover 104 is opened, the processor 162 receives a signal from the safety switch (not shown) indicative of the top cover 104 being open. In response to receiving the signal from the safety switch (not shown), the processor 162 turns off the device 100, which disables the motor 214 to inhibit a user accessing the interior of the housing 102 from being injured while, for example, replacing the resuscitator bag 300.

In some implementations, the device 100 includes a direct current (DC) fan disposed in the housing 102 and attached to one of the first side cover 112 and the second side cover 114. The DC fan circulates air into and out of the housing 102 via the vents 122.

The portable electromechanical resuscitator bag compression device of the present disclosure is a durable, low cost portable device that provides positive pressure ventilation to patients at inhale and exhale rate that corresponds to a desired number of breaths per minute minute to be provided to the patient, while enabling medical personnel to perform other life saving tasks, such as, for example, CPR. The portable electromechanical resuscitator bag compression device of the present disclosure may be used in rugged rural areas where power is limited, or in hospitals where standard respirators are not available or affordable. The shape and size of the portable electromechanical resuscitator bag compression device facilitates transportation of the portable electromechanical resuscitator bag compression device to areas where natural disasters or epidemic have occurred. Additionally, the portable electromechanical resuscitator bag compression device of the present disclosure may also be used as an assisted resuscitation device.

The described embodiments are to be considered in all respects only as illustrative and not restrictive. The scope of the claims should not be limited by the preferred embodiments set forth in the examples, but should be given the broadest interpretation consistent with the description as a whole. All changes that come with meaning and range of equivalency of the claims are to be embraced within their scope.

We claim:

1. A portable electromechanical resuscitator bag compression device comprising:
a housing comprising a first opening and a second opening;
a resuscitator bag disposed at least partially within the housing, the bag comprising an air inlet supported at the first opening of the housing, an air outlet supported at the second opening of the housing, and a self-inflating bag;
a double-sided compression mechanism disposed within the housing, the double sided-compression mechanism:
a pair of arms at least partially surrounding the self-inflating bag, the pair of arms configured to move towards each other to compress the self-inflating bag to provide positive pressure ventilation via the air outlet and to move away from each other to enable re-inflation of the self-inflating bag via the air inlet; and,
a motor coupled to the pair of arms for moving the pair of arms towards and away from each other.

2. The portable electromechanical resuscitator bag compression device of claim 1, wherein the self-inflating bag floats in between the pair of arms of the double-sided compression mechanism.

3. The portable electromechanical resuscitator bag compression device of claim 1, wherein a first arm of pair of arms faces a first side of the self-inflating bag and a second arm of the pair or arms faces a second side of the self-inflating bag.

4. The portable electromechanical resuscitator bag compression device of claim 3, wherein the first arm comprises a first layer of frictionless material for reducing wear on the first side of the self-inflating bag and the second arm comprises a second layer of frictionless material.

5. The portable electromechanical resuscitator bag compression device of claim 3, wherein the first arm has a cam shape to reduce wear on the first side of the self-inflating bag and the second arm has a cam shape to reduce wear on the second side of the self-inflating bag.

6. The portable electromechanical resuscitator bag compression device of claim 3, wherein the first arm includes a first hook coupled to a first hoop on the first side of the self-inflating bag and the second arm includes a second hook coupled to a second hoop on the second side of the self-inflating bag for controlling re-inflation of the self-inflating bag when the first arm and the second arm move away from each other.

7. The portable electromechanical resuscitator bag compression device of claim 1, further comprising:
an input device for inputting an inhale and exhale rate for the positive pressure ventilation.

8. The portable electromechanical resuscitator bag compression device of claim 1, further comprising:
a processor in communication with the input device and configured to:
receive the inhale and exhale rate from the input device;
determine a rate of compression for the double-sided compression mechanism corresponding to the inhale and exhale rate;
control the motor for controlling movement of the pair of arms towards and away from each other at a rate of compression corresponding to the inhale and exhale rate to provide positive pressure ventilation via the air outlet at a number of breaths per minute corresponding to the inhale and exhale rate.

9. The portable electromechanical resuscitator bag compression device of claim 3, wherein the first arm comprises a first pressure sensor for measuring a force applied to the first side of the self-inflating bag and the second arm comprises a second pressure sensor for measuring a force applied to the second side of the self-inflating bag as the pair of arms move towards and away from each other.

10. The portable electromechanical resuscitator bag compression device of claim 1, further comprising:
a power supply disposed in the housing for supplying power to the processor and the motor.

11. The portable electromechanical resuscitator bag compression device of claim 10, wherein the portable electromechanical resuscitator bag compression device further comprises a power switch coupled to the power supply for connecting and disconnecting the power supplied by the power supply to the processor and the motor.

12. The portable electromechanical resuscitator bag compression device of claim 1, wherein the housing comprises:
a front cover comprising a handle for lifting the portable electromechanical resuscitator bag compression device; and
a back cover comprising a recess configured for grasping by a hand of a user to facilitate lifting of the portable electromechanical resuscitator bag compression device.

13. The portable electromechanical resuscitator bag compression device claim 12, wherein a first side cover of the pair of side covers comprises vents for circulating air into the housing and for dissipating heat from within the housing.

14. The portable electromechanical resuscitator bag compression device of claim 12, further comprising:
a strap coupled to each side cover and extending over the top cover to facilitate carrying the portable electromechanical resuscitator bag compression device.

15. The portable electromechanical resuscitator bag compression device of claim 1, further comprising an output device coupled to the processor for providing an audible output when operation of the portable electromechanical resuscitator bag compression device fails.

16. The portable electromechanical resuscitator bag compression device of claim 1, further comprising:
a removable battery disposed in the housing for supplying power to the processor and the motor.

17. The portable electromechanical resuscitator bag compression device of claim 10, further comprising:
a power switch for activating and deactivating the power supply to discontinue supplying power to the processor and the motor.
